(19) United States
(12) Patent Application Publication
(10) Pub. No.: US 2026/0182943 A1
(43) Pub. Date: Jul. 2, 2026

(54) IMAGING SUPPORT APPARATUS, IMAGING SUPPORT METHOD, IMAGING SUPPORT PROGRAM, AND RADIOGRAPHIC IMAGING APPARATUS

(71) Applicant: FUJIFILM Corporation, Tokyo (JP)

(72) Inventors: Hisatsugu HORIUCHI, Tokyo (JP); Atsushi ONODA, Tokyo (JP); Tsukasa ICHIJO, Tokyo (JP); Takeyasu KOBAYASHI, Tokyo (JP); Moeka KITAMURA, Tokyo (JP)

(73) Assignee: FUJIFILM Corporation, Tokyo (JP)

(21) Appl. No.: 19/430,298

(22) Filed: Dec. 23, 2025

(30) Foreign Application Priority Data
Dec. 27, 2024 (JP) 2024-232511

(51) Int. Cl. A61B 6/46; A61B 6/00; A61B 6/58; G06T 7/593; G06T 11/10

(52) U.S. Cl. CPC A61B 6/463; A61B 6/4405; A61B 6/587; G06T 7/593; G06T 11/10; G06T 2207/10016; G06T 2207/10024; G06T 2207/10028; G06T 2207/20224; G06T 2210/41

(57) ABSTRACT

An imaging support apparatus includes a display, and a processor, in which the processor is configured to: acquire distance information representing an imaging distance in a direction from a radiation source toward a subject; acquire an optical image in the direction from the radiation source toward the subject; derive a difference distance image in which a display mode of each pixel differs in accordance with a difference between the imaging distance and a target distance; and superimpose the difference distance image on the optical image to display a superimposed image on a display.

### CROSS-REFERENCE TO RELATED APPLICATION

The present application claims priority from Japanese Patent Application No. 2024-232511, filed on Dec. 27, 2024, the entire disclosure of which is incorporated herein by reference.

### BACKGROUND

### Technical Field

The present disclosure relates to an imaging support apparatus, an imaging support method, an imaging support program, and a radiographic imaging apparatus.

### Related Art

A radiation image of a patient is captured using a radiation detector at a ward-round destination using a mobile radiographic imaging apparatus (ward-round cart). In a case where the radiographic imaging is performed at the ward-round destination in this way, it is required to align a radiation source and the radiation detector. Specifically, it is required to adjust a distance, a relative position, a relative angle between the radiation source and the radiation detector such that a source to image receptor distance (SID), which is a spacing between the radiation source and the radiation detector, matches a target distance, a center of radiation emitted from the radiation source matches an imaging center of a subject, and an optical axis of the radiation intersects the radiation detector perpendicularly. Therefore, a method of displaying information required for alignment, such as the SID, an angle of the radiation source, and an angle of the radiation detector, on a display provided in a radiographic imaging apparatus to support the alignment has been proposed (see, for example, JP2023-116868A). The angle of the radiation source and the angle of the radiation detector are angles about two axes such as a pitch angle and a roll angle.

However, in a case where the ward-round cart is installed obliquely with respect to a patient table, two axes for adjusting the angle of the radiation source and two axes for adjusting the angle of the radiation detector do not match. In this case, it is not possible to easily check whether the angle of the radiation source and the angle of the radiation detector are aligned simply by displaying the angle.

### SUMMARY OF THE INVENTION

The present disclosure has been made in view of the above-described circumstances, and an object thereof is to easily perform angular alignment between the radiation source and the radiation detector.

The present disclosure relates to an imaging support apparatus for a radiographic imaging apparatus including a radiation source that emits radiation, the imaging support apparatus comprising: a display; and a processor, in which the processor is configured to: acquire distance information representing an imaging distance in a direction from the radiation source toward a subject; acquire an optical image in the direction from the radiation source toward the subject; derive a difference distance image in which a display mode of each pixel differs in accordance with a difference between the imaging distance and a target distance; and superimpose the difference distance image on the optical image to display a superimposed image on the display.

In the imaging support apparatus according to the present disclosure, the display mode may be at least one of a color, a density, or a pattern.

In the imaging support apparatus according to the present disclosure, the display may be mounted on a radiation source unit including the radiation source.

In the imaging support apparatus according to the present disclosure, the processor may be configured to derive the difference distance image within a predetermined first distance range based on the target distance.

In the imaging support apparatus according to the present disclosure, the processor may be configured to derive the difference distance image in which a display mode of a distance corresponding to the target distance is different from display modes of other distances.

In the imaging support apparatus according to the present disclosure, the processor may be configured to set the target distance in accordance with an imaging menu designated by a user.

In the imaging support apparatus according to the present disclosure, the processor may be configured to further display the target distance on the display, and match a display mode of a display region of the target distance to a display mode of a representative value of the difference in the difference distance image.

In the imaging support apparatus according to the present disclosure, the processor may be configured to detect a plane in the optical image based on the imaging distance, and derive the difference distance image on the plane.

In the imaging support apparatus according to the present disclosure, the processor may be configured to detect a subject region from the optical image, and derive the difference distance image in a region other than the subject region.

In the imaging support apparatus according to the present disclosure, the processor may be configured to extract a region of a radiation detector that detects radiation transmitted through the subject from the optical image, and derive the difference distance image in the region of the radiation detector.

The present disclosure relates to an imaging support method for a radiographic imaging apparatus including a radiation source that emits radiation, the imaging support method being executed by a computer, the imaging support method comprising: acquiring distance information representing an imaging distance in a direction from the radiation source toward a subject; acquiring an optical image in the direction from the radiation source toward the subject; deriving a difference distance image in which a display mode of each pixel differs in accordance with a difference between the imaging distance and a target distance; and superimposing the difference distance image on the optical image to display a superimposed image on a display.

The present disclosure relates to an imaging support program for a radiographic imaging apparatus including a radiation source that emits radiation, the imaging support program causing a computer to execute: a procedure of acquiring distance information representing an imaging distance in a direction from the radiation source toward a subject; a procedure of acquiring an optical image in the direction from the radiation source toward the subject; a procedure of deriving a difference distance image in which a display mode of each pixel differs in accordance with a difference between the imaging distance and a target distance; and a procedure of superimposing the difference distance image on the optical image to display a superimposed image on a display.

It should be noted that the disclosed technology may be applied to a program product.

The present disclosure relates to a radiographic imaging apparatus comprising: a radiation source; a sensor that acquires distance information representing an imaging distance in a direction from the radiation source toward a subject; an optical camera that captures an optical image in the direction from the radiation source toward the subject; and the imaging support apparatus according to the present disclosure.

The radiographic imaging apparatus according to the present disclosure may further comprise: a body that is movable; and an arm that is foldable and that connects the body to the radiation source.

In the radiographic imaging apparatus according to the present disclosure, information representing a depth in accordance with the display mode in the difference distance image may be applied to the radiation source, the arm, and the body.

In the radiographic imaging apparatus according to the present disclosure, the display mode may be a color, and a color representing a depth of the imaging distance may be applied to the radiation source, the arm, and the body.

According to the present disclosure, angular alignment between the radiation source and the radiation detector can be easily performed.

### BRIEF DESCRIPTION OF THE DRAWINGS

FIG. 1 is an external perspective view of a radiographic imaging apparatus to which an imaging support apparatus according to the present embodiment is applied.

FIG. 2 is a diagram illustrating a state where the radiographic imaging apparatus according to the present embodiment is used.

FIG. 3 is a diagram illustrating a detailed configuration of a radiation source unit.

FIG. 4 is a diagram illustrating a hardware configuration of a computer for executing processing of the imaging support apparatus according to the present embodiment.

FIG. 5 is a functional block diagram of the imaging support apparatus according to the present embodiment.

FIG. 6 is a diagram illustrating plane detection.

FIG. 7 is a diagram illustrating a display screen displayed on a display.

FIG. 8 is a diagram illustrating a state where the radiation source unit is tilted to a right side of a subject on a patient table.

FIG. 9 is a diagram illustrating a state where the radiation source unit is tilted to a left side of the subject on the patient table.

FIG. 10 is a diagram illustrating a display screen displayed on the display.

FIG. 11 is a diagram illustrating a state where the radiation source unit is tilted to a head side of the subject on the patient table.

FIG. 12 is a diagram illustrating a display screen displayed on the display.

FIG. 13 is a diagram illustrating a state where the radiation source unit is tilted to a leg side of the subject on the patient table.

FIG. 14 is a diagram illustrating a display screen displayed on the display.

FIG. 15 is a diagram illustrating a display screen displayed on the display.

FIG. 16 is a flowchart illustrating processing performed in the present embodiment.

FIG. 17 is a diagram illustrating a state where an arm of the radiographic imaging apparatus is extended obliquely relative to the patient table.

FIG. 18 is a diagram illustrating detection of a region of a radiation detector.

FIG. 19 is a diagram illustrating a display screen displayed on the display.

FIG. 20 is a diagram illustrating the radiographic imaging apparatus in which a member is colored in accordance with a distance.

### DETAILED DESCRIPTION

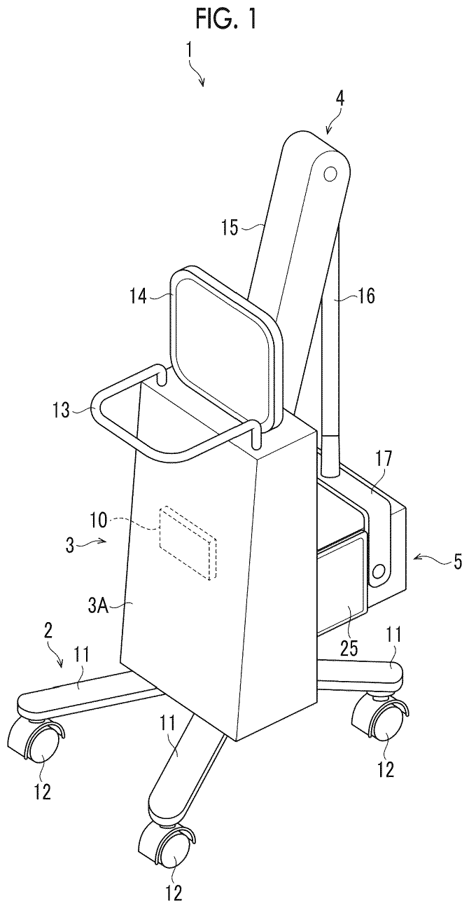

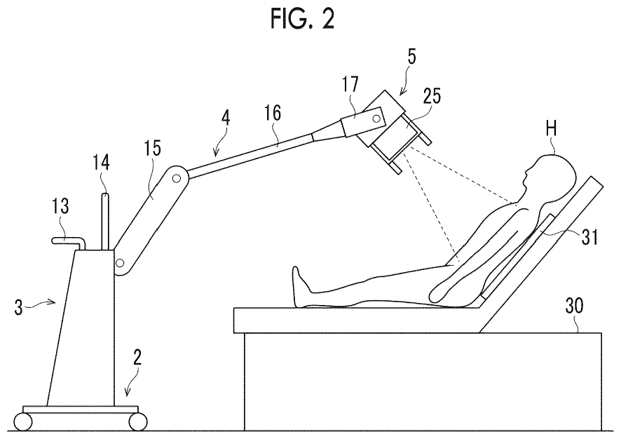

Hereinafter, an embodiment of the present disclosure will be described with reference to the accompanying drawings. FIG. 1 is an external perspective view of a radiographic imaging apparatus to which an imaging support apparatus according to the present embodiment is applied, and FIG. 2 is a diagram illustrating a state where the radiographic imaging apparatus according to the present embodiment is used. A radiographic imaging apparatus 1 to which the imaging support apparatus according to the present embodiment is applied is a ward-round cart type radiographic imaging apparatus, and includes a leg part 2 that is movable on an apparatus placement surface, a body 3 that is supported on the leg part 2, an arm 4 that is connected to the body 3, and a radiation source unit 5 that is mounted on a distal end portion of the arm 4.

The leg part 2 includes four legs 11 and wheel parts 12 mounted on lower surfaces of distal end portions of the legs 11. A stopper (not illustrated) is provided in the wheel part 12 such that the wheels do not rotate unintentionally.

The body 3 accommodates a computer 10, a battery, and the like for controlling the radiographic imaging apparatus 1 in a housing 3A. The computer 10 includes the imaging support apparatus according to the present embodiment. A handle 13 for pushing or pulling the radiographic imaging apparatus is mounted on an upper end of the housing 3A. An operation panel 14 is mounted on an upper portion of the housing 3A.

As the operation panel 14, a touch panel type is adopted in which a display is integrated, and the operation panel 14 receives an instruction of an operator, such as setting of imaging conditions and imaging start, and inputs the instruction to the radiographic imaging apparatus 1. As an imaging menu, chest imaging, extremity imaging, upper-body imaging, and the like can be set.

The arm 4 consists of a first member 15 and a second member 16 that are foldable. The first member 15 is connected to the body 3 so as to be rotatable in an up-down direction. The first member 15 and the second member 16 are connected so as to be rotatable relative to each other.

The radiation source unit 5 is mounted on a distal end of the second member 16 of the arm 4 by a mounting member 17. The mounting member 17 supports the radiation source unit 5 to be swingable. The mounting member 17 is mounted so as to be rotatable around a major axis of the second member 16.

In a case where the radiographic imaging apparatus 1 is used, for example, as illustrated in FIG. 2, an upper body of a subject H is raised on a patient table 30, and a radiation detector 31 for generating a radiation image in which radiation transmitted through the subject H is detected is inserted between a raised portion of the patient table 30 and the subject H. The operator moves the radiographic imaging apparatus 1 close to the patient table 30, deploys the arm 4 in a folded state, and moves the radiation source unit 5 to perform alignment such that the radiation detector 31 is irradiated with the radiation perpendicularly at the set SID. The alignment of the radiation source unit 5 will be described later.

The radiation detector 31 is a cassette type detector configured to acquire the radiation image of the subject H by detecting the radiation. Further, the radiation detector 31 is a wireless detector, and transmits the radiation image acquired by the irradiation with the radiation to the computer 10 wirelessly.

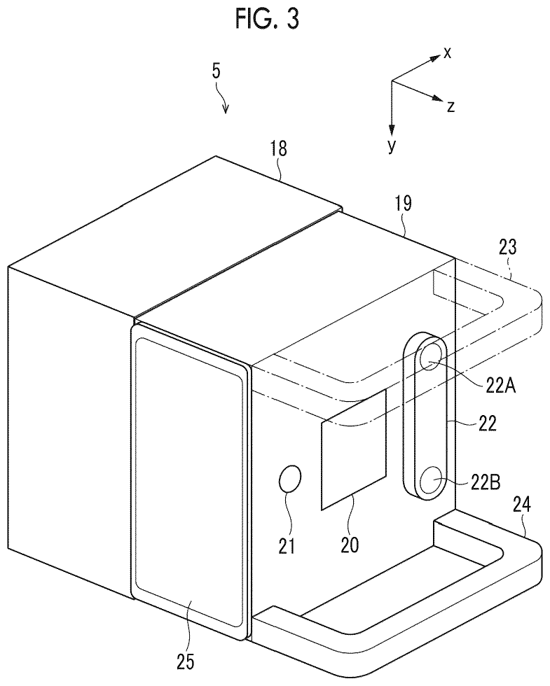

FIG. 3 is a diagram illustrating a detailed configuration of the radiation source unit. As illustrated in FIG. 3, the radiation source unit 5 includes a tube housing part 18 that accommodates a radiation tube such as an X-ray tube, and a collimator 19 that is mounted on the tube housing part 18 so as to be rotatable around an optical axis of the radiation. An emission window 20 for radiation, an optical camera 21, a stereo camera 22, and two handles 23 and 24 are mounted on a radiation emission surface of the collimator 19. A display 25 is mounted on a side surface of the collimator 19. In FIG. 3, the handle 23 is illustrated in phantom for illustrating a configuration of the collimator 19. The display 25 may be mounted on a side surface or a rear surface of the tube housing part 18 instead of the collimator 19.

The collimator 19 sets an irradiation field of the radiation by changing a size of the emission window 20. The irradiation field is set in response to the instruction from the operation panel 14. An irradiation field lamp that is a visible light source is mounted inside the emission window 20 of the collimator 19. By turning on the irradiation field lamp, the subject is irradiated with visible light, and an irradiation range of the visible light changes in accordance with the size of the emission window 20. As a result, the operator can check the radiation irradiation field on the subject H. The collimator 19 is mounted on the tube housing part 18 so as to be rotatable. Therefore, the irradiation field for the subject H can be rotated around the optical axis of the radiation.

The optical camera 21 acquires an optical image G1 in a direction in which the radiation is emitted from the radiation source unit 5. The optical image G1 is a moving image at a predetermined frame rate in which an object on a side irradiated with the radiation from the radiation source unit 5 is represented by RGB pixels. The acquired optical image G1 is displayed on the display 25 as will be described later. The optical camera 21 is configured to acquire a color optical image G1, but may acquire a monochrome optical image G1.

The stereo camera 22 includes two cameras 22A and 22B, and acquires an imaging distance image G2 by measuring a distance based on the principle of triangulation. The imaging distance image G2 is also a moving image at a predetermined frame rate. In the imaging distance image G2, each pixel represents an imaging distance in a direction from the radiation source unit 5 toward the subject. A time-of-flight (TOF) camera that measures a distance by a time for light to return may be used instead of the stereo camera 22. The imaging distance image may be derived by using a light detection and ranging (LiDAR) sensor. The stereo camera 22, the TOF camera, and the LiDAR sensor are examples of a sensor that acquires distance information representing an imaging distance according to the present disclosure. The imaging distance image G2 is an example of distance information representing an imaging distance according to the present disclosure. The distance information is not limited to an image format, and may be a numerical value representing the imaging distance itself.

The handles 23 and 24 are used by the operator to grip and adjust a position and an angle of the radiation source unit 5. Here, in a case where an x-axis, a y-axis, and a z-axis are set as illustrated in FIG. 3, the collimator 19 is mounted on the tube housing part 18 so as to be rotatable around the z-axis. Therefore, the irradiation field of the X-rays can be rotated. In addition, as illustrated in FIG. 1, the radiation source unit 5 is mounted on the second member 16 so as to be rotatable around the major axis of the second member 16 and is mounted on the second member 16 so as to be swingable by the mounting member 17. Therefore, the angles around the x-axis and the y-axis illustrated in FIG. 3 can be adjusted.

The optical image G1 captured by the optical camera 21 is displayed on the display 25. The display content on the display 25 will be described later.

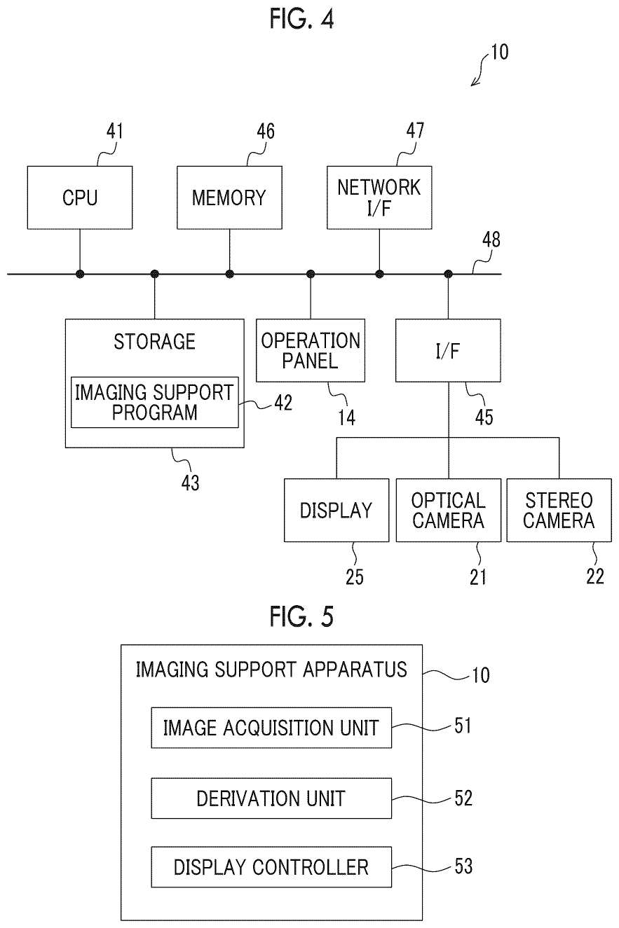

Hereinafter, the computer for executing processing of the imaging support apparatus according to the present embodiment will be described. FIG. 4 is a diagram illustrating a hardware configuration of the computer for executing the processing of the imaging support apparatus. As illustrated in FIG. 4, the computer 10 includes a central processing unit (CPU) 41, a non-volatile storage 43, and a memory 46 as a temporary storage area. In addition, the computer 10 includes the operation panel 14, a network interface (I/F) 47 that is connected to a network (not illustrated), and a wired and wireless I/F 45 for connecting the optical camera 21, the stereo camera 22, and the display 25 to the computer 10. The CPU 41, the storage 43, the operation panel 14, the I/F 45, the memory 46, and the network I/F 47 are connected to a bus 48. The CPU 41 is an example of a processor according to the present disclosure.

The computer 10 performs processing of displaying the radiation image acquired by the radiation detector 31 and transmitting the radiation image to an external apparatus or the like, but detailed description of these types of processing will be omitted here. Further, the computer 10 includes the imaging support apparatus according to the present embodiment. Therefore, in the following description, the imaging support apparatus according to the present embodiment will also be denoted by reference numeral 10.

The storage 43 is implemented by a hard disk drive (HDD), a solid state drive (SSD), a flash memory, and the like. An imaging support program 42 is stored in the storage 43 as a storage medium. The CPU 41 reads out the imaging support program 42 from the storage 43, loads the readout imaging support program 42 into the memory 46, and executes the loaded imaging support program 42.

Hereinafter, a functional configuration of the imaging support apparatus according to the present embodiment will be described. FIG. 5 is a diagram illustrating the functional configuration of the imaging support apparatus according to the present embodiment. As illustrated in FIG. 5, the imaging support apparatus 10 includes an image acquisition unit 51, a derivation unit 52, and a display controller 53. In a case where the CPU 41 executes the imaging support program 42, the CPU 41 functions as the image acquisition unit 51, the derivation unit 52, and the display controller 53.

The image acquisition unit 51 acquires the optical image G1 acquired by the optical camera 21, and the imaging distance image G2 acquired by the stereo camera 22.

The derivation unit 52 derives a difference distance image in which a display mode of each pixel differs in accordance with the difference between the imaging distance and the target distance. Here, the target distance is an SID, which is a spacing between the radiation source unit 5 and the radiation detector 31. The SID may be set by the operator by a numerical value from the operation panel 14, or may be automatically set in accordance with an imaging menu registered in advance in the radiographic imaging apparatus 1. For example, an SID of 100 cm is registered in a case where the imaging menu is portable chest imaging, an SID of 180 cm is registered in a case where the imaging menu is upright chest imaging, an SID of 100 cm is registered in a case where the imaging menu is extremity imaging, and an SID of 120 cm is registered in a case where the imaging menu is hip joint imaging. In this case, in a case where the operator designates the imaging menu from the operation panel 14, an appropriate SID corresponding to the imaging menu is set as the target distance.

The derivation unit 52 derives a difference between the imaging distance represented by each pixel of the imaging distance image G2 and the target distance. Then, a difference distance image Gs is derived in which each pixel has a display mode corresponding to the difference. In the present embodiment, the derivation unit 52 derives the difference distance image Gs in which each pixel has a color corresponding to the difference. In the present embodiment, in a case where the difference from the target distance is ±5 cm, it is regarded as matching the target distance, and the derivation unit 52 derives the difference distance image Gs in which the color of the pixel matching the target distance is distinguished from the color of the other pixels. For example, light yellow can be used as the color matching the target distance. In this case, light yellow is a color representing that the imaging distance matches the target distance.

In the present embodiment, the difference is derived by subtracting the imaging distance from the target distance. Therefore, in a case where the difference is a positive value, the object at the imaging distance is located at a position closer to the radiation source unit 5 than the target distance. On the other hand, in a case where the difference is a negative value, the object at the imaging distance is located at a position farther from the radiation source unit 5 than the target distance.

In addition, in a case where the difference from the target distance is greater than 5 cm, that is, in a case where the imaging distance is closer to the radiation source unit 5 than the target distance, the derivation unit 52 sets the color corresponding to the difference distance image Gs to a cool color. For example, in a case where the difference from the target distance is greater than 5 cm and 10 cm or less, in a case where the difference from the target distance is greater than 10 cm and 15 cm or less, and in a case where the difference from the target distance is greater than 15 cm and 20 cm or less, the difference distance image Gs is derived such that the color becomes gradually more bluish.

In addition, in a case where the difference from the target distance is less than −5 cm, that is, in a case where the imaging distance is farther from the radiation source unit 5 than the target distance, the derivation unit 52 sets the color corresponding to the difference distance image Gs to a warm color. For example, in a case where the difference from the target distance is less than −5 cm and −10 cm or less, in a case where the difference from the target distance is less than −10 cm and −15 cm or less, and in a case where the difference from the target distance is less than −15 cm and −20 cm or less, the difference distance image Gs is derived such that the color becomes gradually reddish.

In addition, the derivation unit 52 may derive the difference distance image Gs only within a predetermined distance range based on the target distance. For example, the difference distance image Gs may be derived only in a range of ±25 cm based on the target distance. Here, ±25 cm is an example of a first distance according to the present disclosure.

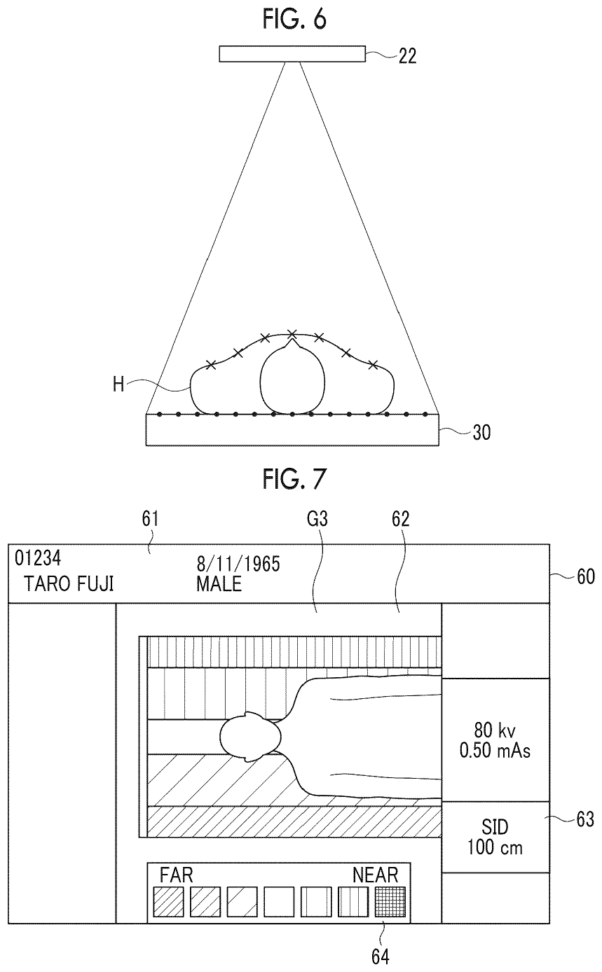

In addition, the derivation unit 52 may detect a plane in the imaging distance image G2, and derive the difference distance image Gs only on the detected plane. FIG. 6 is a diagram illustrating plane detection. As illustrated in FIG. 6, for a distance within a certain angle of view from the stereo camera 22 near the subject H on the patient table 30, the surface of the patient table 30 is flat and has a certain area, so that the imaging distances in the plurality of pixels are within a predetermined range, and a pixel group (indicated by black circles in FIG. 6) in which the imaging distances are within the predetermined range has an area equal to or larger than a certain value. On the other hand, the surface of the subject H is curved, and thus the imaging distances in the plurality of pixels (indicated by ×marks in FIG. 6) representing the surface of the subject H exceed the predetermined range. In addition, the predetermined range can be, for example, ±2 cm.

Therefore, in a case where the imaging distances in the plurality of pixels are within the predetermined range and the pixel group in which the imaging distances are within the predetermined range has an area equal to or larger than a certain value in the imaging distance image G2, the derivation unit 52 determines that the pixel group constitutes the plane. The derivation unit 52 may derive the difference distance image Gs only on the determined plane. Since the pixel group in the predetermined range is determined to be the plane in a case where the pixel group has an area equal to or larger than a certain value, it is possible to prevent the detection of a small object having a flat surface as the plane in a case where the small object is near the patient table 30.

In addition, the derivation unit 52 may detect the subject H in the optical image G1, and derive the difference distance image Gs in a region other than the subject H in the optical image G1. For the detection of the region of the subject H in the optical image G1, for example, a trained model that has been trained through machine learning to detect the subject region may be used.

In addition, the derivation unit 52 may derive the difference distance image Gs only on the determined plane in the region other than the subject H in the optical image G1. Hereinafter, in the present embodiment, the derivation unit 52 derives the difference distance image Gs only on the determined plane in the region other than the subject H in the optical image G1.

The display controller 53 superimposes the difference distance image Gs derived by the derivation unit 52 on the optical image G1, and displays a superimposed image on the display 25. FIG. 7 is a diagram illustrating a display screen displayed on the display 25. As illustrated in FIG. 7, a display screen 60 includes an information region 61 that displays information on the subject H, an image region 62 that displays an image, a condition region 63 that displays imaging conditions such as the SID, and a color bar 64 that indicates the color corresponding to the difference distance.

A name, a patient number, a birthday, a gender, and the like of the subject H are displayed in the information region 61. A superimposed image G3 in which the difference distance image Gs is superimposed on the optical image G1 is displayed in the image region 62. In the condition region 63, a tube voltage (for example, 80 kV), an mAs value (for example, 0.50 mAs), and the SID (100 cm) are displayed. In the color bar 64, patches of colors representing the distance corresponding to the difference in the difference distance image Gs are arranged in order of the distance. In FIG. 7, for the sake of description, the difference in the color is indicated by different patterns in the patches of the color bar 64. In the color bar 64, labels “far” and “near” are assigned to easily understand whether the displayed color is far or near from the target distance.

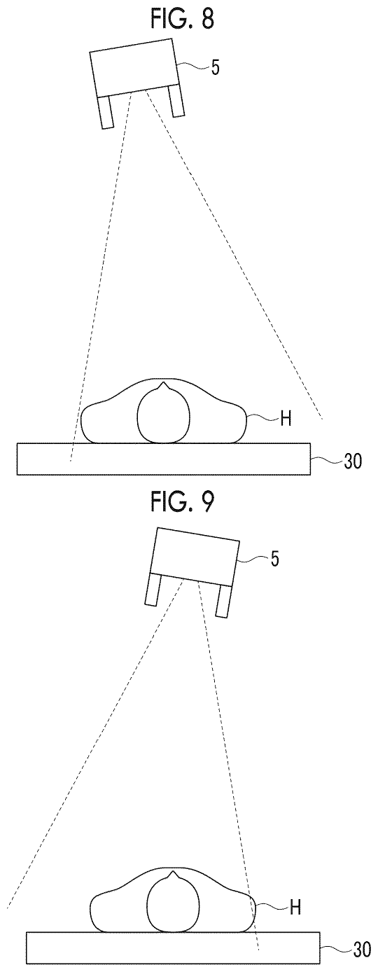

Here, as illustrated in FIG. 8, in a case where the radiation source unit 5 is tilted to the right side of the subject H on the patient table 30, the right side of the subject H is farther from the radiation source unit 5, and the left side of the subject H is closer to the radiation source unit 5. Therefore, as illustrated in FIG. 7, in the superimposed image G3, a warm color indicating that the right side of the subject H is located at a position farther from the radiation source unit 5 than the target distance (that is, the difference is a negative value) is displayed on the patient table 30, and a cool color indicating that the left side of the subject H is located at a position closer to the radiation source unit 5 than the target distance (that is, the difference is a positive value) is displayed on the patient table 30.

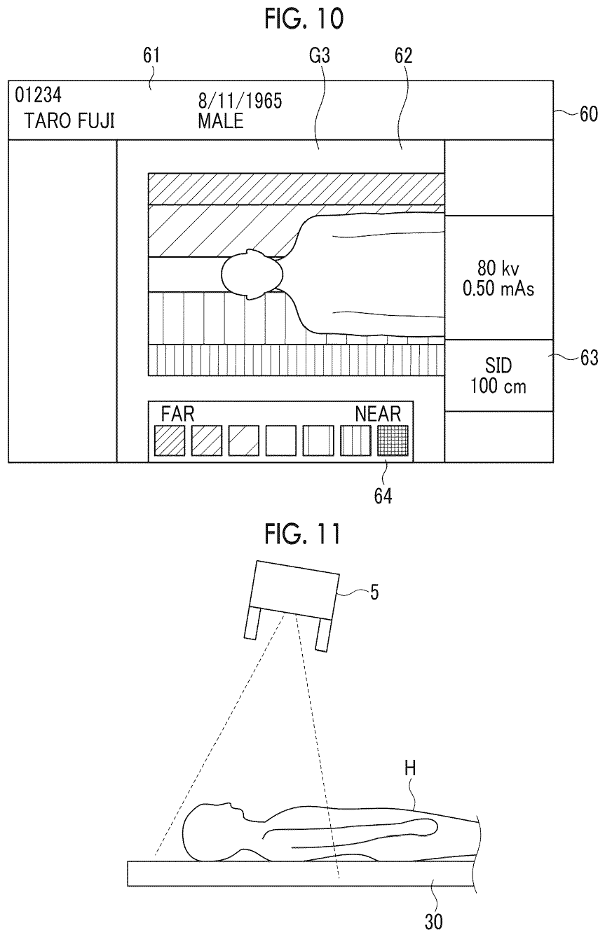

In addition, as illustrated in FIG. 9, in a case where the radiation source unit 5 is tilted to the left side of the subject H on the patient table 30, the left side of the subject H is farther from the radiation source unit 5, and the right side of the subject H is closer to the radiation source unit 5. Therefore, as illustrated in FIG. 10, in the superimposed image G3, a warm color indicating that the left side of the subject H is located at a position farther from the radiation source unit 5 than the target distance (that is, the difference is a negative value) is displayed on the patient table 30, and a cool color indicating that the right side of the subject H is located at a position closer to the radiation source unit 5 than the target distance (that is, the difference is a positive value) is displayed on the patient table 30.

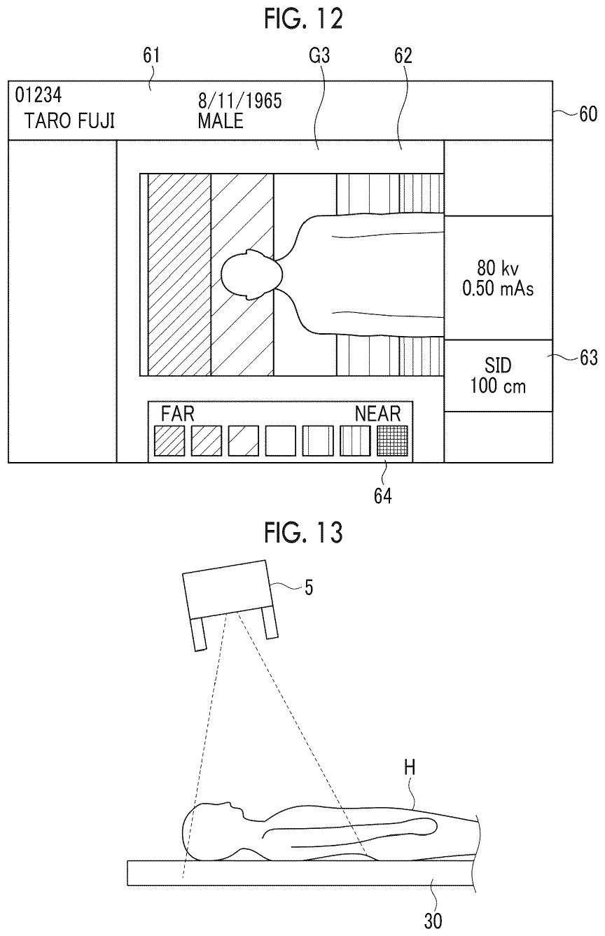

In addition, as illustrated in FIG. 11, in a case where the radiation source unit 5 is tilted to the head side of the subject H on the patient table 30, the head side of the subject H is farther from the radiation source unit 5, and the leg side of the subject H is closer to the radiation source unit 5. Therefore, as illustrated in FIG. 12, in the superimposed image G3, a warm color indicating that the head side of the subject H is located at a position farther from the radiation source unit 5 than the target distance (that is, the difference is a negative value) is displayed on the patient table 30, and a cool color indicating that the leg side of the subject H is located at a position closer to the radiation source unit 5 than the target distance (that is, the difference is a positive value) is displayed on the patient table 30.

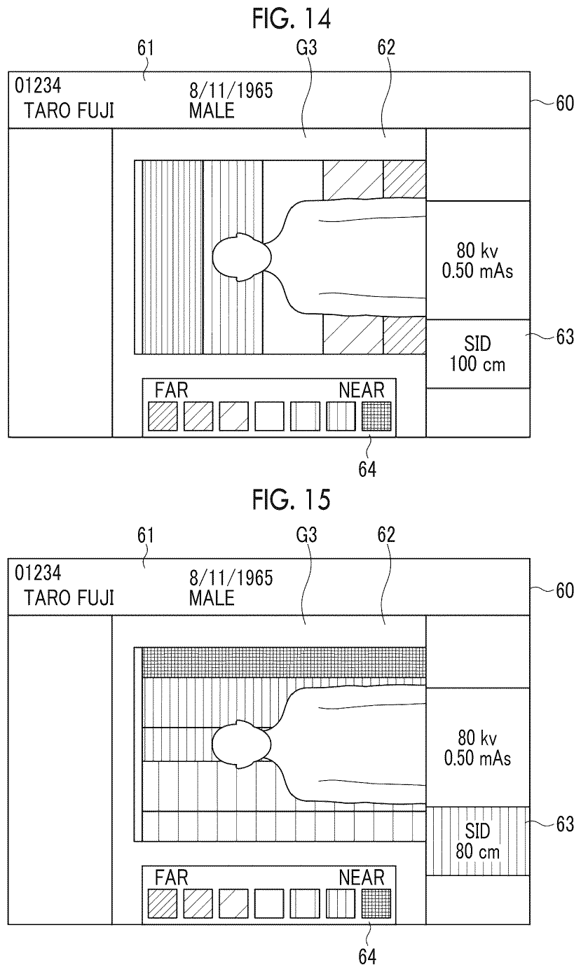

In addition, as illustrated in FIG. 13, in a case where the radiation source unit 5 is tilted to the leg side of the subject H on the patient table 30, the leg side of the subject H is farther from the radiation source unit 5, and the head side of the subject H is closer to the radiation source unit 5. Therefore, as illustrated in FIG. 14, in the superimposed image G3, a warm color indicating that the leg side of the subject H is located at a position farther from the radiation source unit 5 than the target distance (that is, the difference is a negative value) is displayed on the patient table 30, and a cool color indicating that the head side of the subject H is located at a position closer to the radiation source unit 5 than the target distance (that is, the difference is a positive value) is displayed on the patient table 30.

In the examples illustrated in FIGS. 7, 10, 12, and 14, since the superimposed image G3 includes a portion at the target distance relative to the SID (target distance), the superimposed image G3 includes a region in which a color indicating a ±5 cm difference between the imaging distance and the target distance is displayed. On the other hand, in a state illustrated in FIG. 8, in a case where the radiation source unit 5 is, relative to the patient table 30 as a whole, at a position closer than the SID or farther than the SID, the superimposed image G3 may no longer display the color indicating a ±5 cm difference between the imaging distance and the target distance. In this case, a background color of a region (referred to as an SID region) in which the SID of the condition region 63 is displayed may be changed in accordance with the value of the difference in the difference distance image Gs. For example, as illustrated in FIG. 15, in a case where an average value of the difference values in the difference distance image Gs is 80 cm, the color may be changed to a color representing a distance that is 20 cm closer to 100 cm that is the target value. Instead of the color of the SID region, a color of a numerical value of the SID displayed in the SID region may be changed, or a color of a frame of the SID region may be changed.

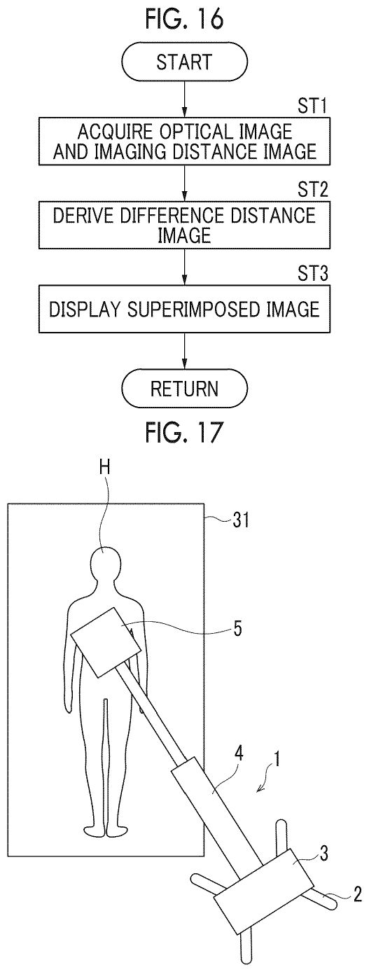

Hereinafter, processing performed in the present embodiment will be described. FIG. 16 is a flowchart illustrating the processing performed in the present embodiment. First, the image acquisition unit 51 acquires the optical image G1 acquired by the optical camera 21, and the imaging distance image G2 acquired by the stereo camera 22 (step ST1). The derivation unit 52 derives the difference distance image Gs in which the color differs in accordance with the difference from the target distance by deriving the difference from the target distance for each pixel of the imaging distance image G2 (step ST2). The display controller 53 displays the superimposed image G3 in which the difference distance image Gs is superimposed on the optical image G1 on the display 25 (step ST3), and returns to step ST1.

As described above, in the present embodiment, the difference distance image Gs in which the color differs in accordance with the difference from the target distance is derived, the difference distance image Gs is superimposed on the optical image G1, and the superimposed image is displayed on the display 25. Therefore, the operator can adjust the position of the radiation source unit 5 and the angle of the radiation source unit 5 relative to the patient table 30 while viewing the superimposed image G3 displayed on the display 25 to perform the alignment of the radiation source unit 5 based on the difference in the color.

Here, the radiation source unit 5 is mounted so as to be rotatable and swingable around the major axis of the second member 16 of the arm 4 (around the z-axis illustrated in FIG. 3). Therefore, as illustrated in FIG. 17, in a case where there is no space around the patient table 30 and the arm 4 of the radiographic imaging apparatus 1 needs to be extended obliquely relative to the patient table 30, it is difficult to determine whether the radiation source unit 5 is aligned to face the subject H directly simply by displaying the angle of the radiation source unit 5 on the operation panel 14.

In the present embodiment, the operator can perform the alignment of the radiation source unit 5 in a sensory manner based on the difference in the color in the superimposed image G3 displayed on the display 25. Therefore, the operation of aligning the radiation source unit 5 can be easily performed.

After the alignment, the operator operates the operation panel 14 to turn on the irradiation field lamp and irradiates the subject H with the visible light representing the irradiation field. Then, after the irradiation field is adjusted, an instruction to perform the imaging is issued from the operation panel 14, the imaging of the subject H is performed, and thus the radiation image is acquired.

In the above-described embodiment, the derivation unit 52 detects the plane in the imaging distance image G2 and derives the difference distance image Gs only in the detected plane, but the present disclosure is not limited to this. The difference distance image Gs may be derived for an entire region of the imaging distance image G2.

Further, in the above-described embodiment, the difference distance image Gs is derived in the predetermined range based on the target distance, but the present disclosure is not limited to this. The difference distance image Gs may be derived for all distances.

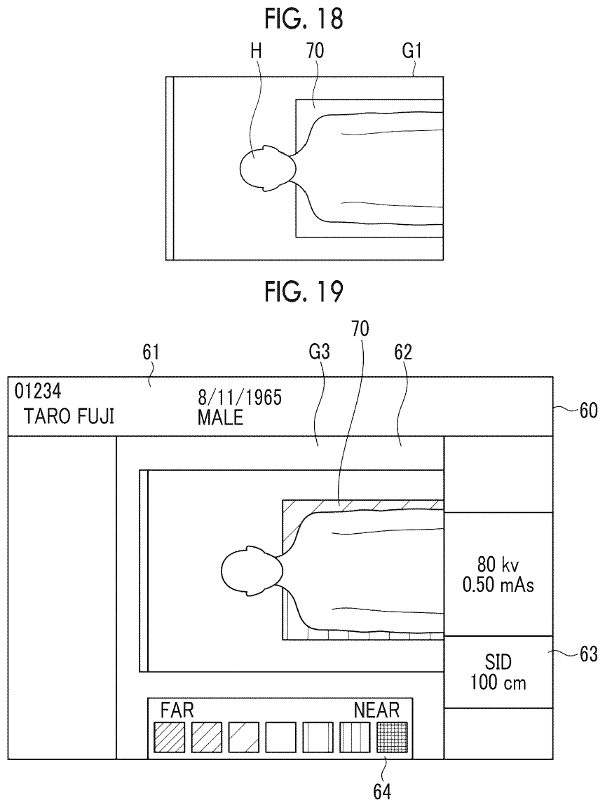

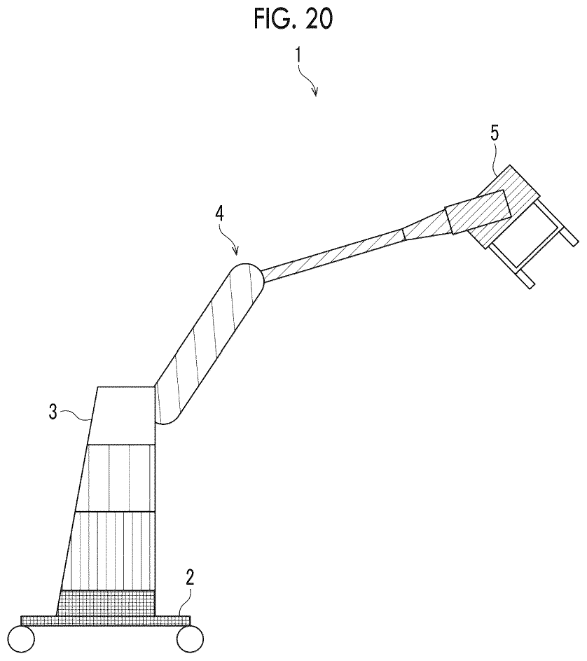

Furthermore, in the above-described embodiment, the region of the radiation detector 31 included in the optical image G1 may be detected, and the difference distance image Gs may be derived only in the region of the radiation detector 31. In this case, the detection of the region of the radiation detector 31 may be performed by using a detection model constructed by machine learning using images of a plurality of radiation detectors 31 as training data. As a result, as illustrated in FIG. 18, a region 70 of the radiation detector included in the optical image G1 can be detected. In this case, as illustrated in FIG. 19, the difference distance image Gs may be derived only in the region 70 of the radiation detector and may be superimposed on the optical image G1, and the superimposed image may be displayed on the display 25.

Further, in the above-described embodiment, the color bar 64 is always displayed on the display 25, but the present disclosure is not limited to this. Displaying the color bar 64 and hiding the color bar 64 may be switched on the display screen such that the color bar 64 can be referred to only when required. In addition, as illustrated in FIG. 20, the member constituting the radiographic imaging apparatus 1 may be colored in accordance with the distance instead of displaying the color bar 64. For example, the upper portion of the body 3 may be colored light yellow, with the color stepwise becoming more bluish from the upper portion of the body 3 toward the arm 4 and the radiation source unit 5, and stepwise becoming more reddish from the upper portion of the body 3 toward the lower portion and further toward the leg part 2.

Furthermore, in the above-described embodiment, the difference from the target distance is represented by the difference in the color in the color bar 64, but the present disclosure is not limited to this. The difference from the target distance may be represented by a difference in density of the same color. For example, the density may be relatively decreased in a case where the difference from the target distance is a positive value, and the density may be relatively increased in a case where the difference is a negative value. In this case, the “same color” includes monochrome.

Further, the difference from the target distance may be represented by a difference in pattern instead of being limited to the difference in color and the difference in density. Furthermore, the difference from the target distance may be represented by a combination of two or more of the color, the density, or the pattern.

In the above-described embodiment, the radiation source unit 5 includes the optical camera 21 and the stereo camera 22 as separate bodies, but the present disclosure is not limited to this. A camera may be used in which the optical camera and the stereo camera are built in one housing.

In the present embodiment, each processing is executed by any computer. Also, any computer may execute these processes by a processor as hardware, a program as software, or a combination thereof. In this case, the processor is configured to execute various types of processing in the present embodiment in cooperation with the program and can function as each unit or each means in the present embodiment. Furthermore, the execution order of the processing by the processor is not limited to the above-described order, and may be changed as appropriate. Any computer may be a general-purpose computer, a computer for specific use, a workstation, or another system that can execute each processing.

The processor may be configured by one or more hardware components, and the type of hardware is not limited. For example, the processor may be configured by hardware, such as a central processing unit (CPU), a micro processing unit (MPU), a programmable logic device, such as a field programmable gate array (FPGA), a dedicated circuit that is used to execute specific processing, such as an application-specific integrated circuit (ASIC), a graphics processing unit (GPU), or a neural processing unit (NPU). Furthermore, the type of hardware may be a combination of different types of hardware components. In a case where the plurality of hardware components are configured to execute one or a plurality of types of processing of a certain processor, the plurality of hardware components may be present in devices physically separated from each other or may be present in the same device. Additionally, in any embodiment, the order of each processing by the processor is not limited to the order described above and may be changed as appropriate. In addition, the hardware is configured by an electrical circuit (circuitry) in which circuit elements, such as semiconductor elements, are combined.

Further, the program may be software such as firmware or microcode. Additionally, the program may be, for example, a program module group, and each function thereof may be executed by the processor configured to execute the corresponding function. The program may be a program code or a plurality of code segments stored in one or more non-transitory computer-readable media (for example, storage media or other storages). The program may be distributed and stored across a plurality of non-transitory computer-readable media existing in devices physically separated from each other. The program code or the code segment may represent a procedure, function, subprogram, routine, subroutine, module, software package, class, or any combination of instructions, data structures, or program statements. The program code or the code segment may be connected to another code segment or a hardware circuit by the transmission and reception of information, data, arguments, parameters, or contents in the memory.

In addition, in the above-described embodiment, the imaging support program 42 is stored (installed) in the storage 43 in advance, but the present disclosure is not limited to this. The imaging support program 42 may be provided in a form recorded on a recording medium, such as a compact disc read-only memory (CD-ROM), a digital versatile disc read-only memory (DVD-ROM), and a universal serial bus (USB) memory. In addition, the imaging support program 42 may be downloaded from an external apparatus through the network.

The disclosed technology is applicable to any program product. The program product includes all forms of products for providing the program. For example, the program product includes a program provided through a network such as the Internet, a non-transitory computer-readable recording medium such as a CD-ROM, a DVD, and a USB memory in which the program is stored and the like.

Hereinafter, supplementary notes of the present disclosure are set forth.

### Supplementary Note 1

An imaging support apparatus for a radiographic imaging apparatus including a radiation source that emits radiation, the imaging support apparatus comprising: a display; and a processor, in which the processor is configured to: acquire distance information representing an imaging distance in a direction from the radiation source toward a subject; acquire an optical image in the direction from the radiation source toward the subject; derive a difference distance image in which a display mode of each pixel differs in accordance with a difference between the imaging distance and a target distance; and superimpose the difference distance image on the optical image to display a superimposed image on the display.

### Supplementary Note 2

The imaging support apparatus according to supplementary note 1, in which the display mode is at least one of a color, a density, or a pattern.

### Supplementary Note 3

The imaging support apparatus according to supplementary note 1 or 2, in which the display is mounted on a radiation source unit including the radiation source.

### Supplementary Note 4

The imaging support apparatus according to any one of supplementary notes 1 to 3, in which the processor is configured to derive the difference distance image within a predetermined first distance range based on the target distance.

### Supplementary Note 5

The imaging support apparatus according to any one of supplementary notes 1 to 4, in which the processor is configured to derive the difference distance image in which a display mode of a distance corresponding to the target distance is different from display modes of other distances.

### Supplementary Note 6

The imaging support apparatus according to any one of supplementary notes 1 to 5, in which the processor is configured to set the target distance in accordance with an imaging menu designated by a user.

### Supplementary Note 7

The imaging support apparatus according to any one of supplementary notes 1 to 6, in which the processor is configured to further display the target distance on the display, and match a display mode of a display region of the target distance to a display mode of a representative value of the difference in the difference distance image.

### Supplementary Note 8

The imaging support apparatus according to any one of supplementary notes 1 to 7, in which the processor is configured to detect a plane in the optical image based on the imaging distance, and derive the difference distance image on the plane.

### Supplementary Note 9

The imaging support apparatus according to any one of supplementary notes 1 to 8, in which the processor is configured to detect a subject region from the optical image, and derive the difference distance image in a region other than the subject region.

### Supplementary Note 10

The imaging support apparatus according to any one of supplementary notes 1 to 9, in which the processor is configured to extract a region of a radiation detector that detects radiation transmitted through the subject from the optical image, and derive the difference distance image in the region of the radiation detector.

### Supplementary Note 11

An imaging support method for a radiographic imaging apparatus including a radiation source that emits radiation, the imaging support method being executed by a computer, the imaging support method comprising: acquiring distance information representing an imaging distance in a direction from the radiation source toward a subject; acquiring an optical image in the direction from the radiation source toward the subject; deriving a difference distance image in which a display mode of each pixel differs in accordance with a difference between the imaging distance and a target distance; and superimposing the difference distance image on the optical image to display a superimposed image on a display.

### Supplementary Note 12

An imaging support program for a radiographic imaging apparatus including a radiation source that emits radiation, the imaging support program causing a computer to execute: a procedure of acquiring distance information representing an imaging distance in a direction from the radiation source toward a subject; a procedure of acquiring an optical image in the direction from the radiation source toward the subject; a procedure of deriving a difference distance image in which a display mode of each pixel differs in accordance with a difference between the imaging distance and a target distance; and a procedure of superimposing the difference distance image on the optical image to display a superimposed image on a display.

### Supplementary Note 13

A radiographic imaging apparatus comprising: a radiation source; a sensor that acquires distance information representing an imaging distance in a direction from the radiation source toward a subject; an optical camera that captures an optical image in the direction from the radiation source toward the subject; and the imaging support apparatus according to any one of supplementary notes 1 to 10.

### Supplementary Note 14

The radiographic imaging apparatus according to supplementary note 13, further comprising: a body that is movable; and an arm that is foldable and that connects the body to the radiation source.

### Supplementary Note 15

The radiographic imaging apparatus according to supplementary note 13 or 14, in which information representing a depth in accordance with the display mode in the difference distance image is applied to the radiation source, the arm, and the body.

### Supplementary Note 16

The radiographic imaging apparatus according to supplementary note 15, in which the display mode is a color, and a color representing a depth of the imaging distance is applied to the radiation source, the arm, and the body.

What is claimed is:

1. An imaging support apparatus for a radiographic imaging apparatus including a radiation source that emits radiation, the imaging support apparatus comprising:
a display; and
a processor,
wherein the processor is configured to:
acquire distance information representing an imaging distance in a direction from the radiation source toward a subject;
acquire an optical image in the direction from the radiation source toward the subject;
derive a difference distance image in which a display mode of each pixel differs in accordance with a difference between the imaging distance and a target distance; and
superimpose the difference distance image on the optical image to display a superimposed image on the display.

2. The imaging support apparatus according to claim 1,
wherein the display mode is at least one of a color, a density, or a pattern.

3. The imaging support apparatus according to claim 1,
wherein the display is mounted on a radiation source unit including the radiation source.

4. The imaging support apparatus according to claim 1,
wherein the processor is configured to derive the difference distance image within a predetermined first distance range based on the target distance.

5. The imaging support apparatus according to claim 1,
wherein the processor is configured to derive the difference distance image in which a display mode of a distance corresponding to the target distance is different from display modes of other distances.

6. The imaging support apparatus according to claim 1,
wherein the processor is configured to set the target distance in accordance with an imaging menu designated by a user.

7. The imaging support apparatus according to claim 1,
wherein the processor is configured to further display the target distance on the display, and match a display mode of a display region of the target distance to a display mode of a representative value of the difference in the difference distance image.

8. The imaging support apparatus according to claim 1,
wherein the processor is configured to detect a plane in the optical image based on the imaging distance, and derive the difference distance image on the plane.

9. The imaging support apparatus according to claim 1,
wherein the processor is configured to detect a subject region from the optical image, and derive the difference distance image in a region other than the subject region.

10. The imaging support apparatus according to claim 1,
wherein the processor is configured to extract a region of a radiation detector that detects radiation transmitted through the subject from the optical image, and derive the difference distance image in the region of the radiation detector.

11. An imaging support method for a radiographic imaging apparatus including a radiation source that emits radiation, the imaging support method being executed by a computer, the imaging support method comprising:
acquiring distance information representing an imaging distance in a direction from the radiation source toward a subject;
acquiring an optical image in the direction from the radiation source toward the subject;
deriving a difference distance image in which a display mode of each pixel differs in accordance with a difference between the imaging distance and a target distance; and
superimposing the difference distance image on the optical image to display a superimposed image on a display.

12. A non-transitory computer-readable storage medium that stores an imaging support program for a radiographic imaging apparatus including a radiation source that emits radiation, the imaging support program causing a computer to execute:
a procedure of acquiring distance information representing an imaging distance in a direction from the radiation source toward a subject;
a procedure of acquiring an optical image in the direction from the radiation source toward the subject;
a procedure of deriving a difference distance image in which a display mode of each pixel differs in accordance with a difference between the imaging distance and a target distance; and
a procedure of superimposing the difference distance image on the optical image to display a superimposed image on a display.

13. A radiographic imaging apparatus comprising:
a radiation source;
a sensor that acquires distance information representing an imaging distance in a direction from the radiation source toward a subject;
an optical camera that captures an optical image in the direction from the radiation source toward the subject; and
the imaging support apparatus according to claim 1.

14. The radiographic imaging apparatus according to claim 13, further comprising:
a body that is movable; and
an arm that is foldable and that connects the body to the radiation source.

15. The radiographic imaging apparatus according to claim 14,
wherein information representing a depth in accordance with the display mode in the difference distance image is applied to the radiation source, the arm, and the body.

16. The radiographic imaging apparatus according to claim 15,
wherein the display mode is a color, and
a color representing a depth of the imaging distance is applied to the radiation source, the arm, and the body.
